(19) United States
(12) Patent Application Publication
(10) Pub. No.: US 2025/0058009 A1
(43) Pub. Date: Feb. 20, 2025

(54) EXPLORATION MAPPING AND DISINFECTION ALGORITHM FOR AUTONOMOUS MOBILE ROBOTS WITH UVC LIGHT

(71) Applicants: Kristopher Douglas Rupay, Ottawa (CA); Arash Mahinakbarzadeh, Ottawa (CA); Payam Peter Bahraini, Ottawa (CA)

(72) Inventors: Kristopher Douglas Rupay, Ottawa (CA); Arash Mahinakbarzadeh, Ottawa (CA); Payam Peter Bahraini, Ottawa (CA)

(21) Appl. No.: 18/235,865

(22) Filed: Aug. 20, 2023

(51) Int. Cl. A61L 2/24; A61L 2/10; G01C 21/00; G05D 1/02

(52) U.S. Cl. CPC A61L 2/24; A61L 2/10; G01C 21/3807; A61L 2202/11; A61L 2202/14; A61L 2202/16

(57) ABSTRACT

The present invention introduces an algorithm tailored for autonomous mobile robots equipped with UVC light emission capabilities. This algorithm empowers the robot to deliver a precisely configured level of UVC light energy, facilitating comprehensive surface disinfection within a room without relying on prior spatial knowledge or mapping. The algorithm dynamically learns the environment, guaranteeing the adequate distribution of UVC light through the air to achieve effective surface disinfection. The output of using the algorithm, concludes with a generated heat map, further providing precise evidence of the UVC light levels that have reached the surfaces.

### BACKGROUND

Ultraviolet-C (UVC) light energy is widely recognized for its exceptional pathogen disinfection capabilities. However, the effectiveness of UVC light in disinfecting surfaces poses a challenge due to its limited reflectivity and penetration through various materials.

While numerous exploration algorithms are employed by autonomous mobile robots for spatial mapping, none directly correlate the exploration and mapping process with the optimal distribution of UVC light energy required to disinfect surfaces within a given space, such as a patient room.

Existing autonomous mobile robots that navigate and emit UVC light rely on predetermined maps and fixed paths to determine their movements for efficient disinfection. Unfortunately, this approach demands extensive map programming, particularly when the spatial configuration changes due to object movements.

Furthermore, various stationary UVC-emitting devices, while not classified as autonomous mobile robots, employ measurement sensors to assess room dimensions. However, due to their limited placement positions within a room, these devices fail to ensure comprehensive UVC light coverage on all surfaces. Human error further compounds the issue, as consistent device placement cannot be guaranteed across operations. Additionally, the angle of light incidence, a critical determinant of energy delivery to surfaces, is often disregarded. For this style of UVC-emitting device to be effective, multiple placement positions are required within a room, to reach the various angles and proximity to surfaces. This extra movement, further adds complexity, and unnecessary time for operators.

Leveraging established principles of light energy projection onto surfaces, it becomes possible to calculate and monitor the UVC light energy reaching various surfaces. By integrating these calculations, the algorithm optimizes robotic movements to achieve effective space disinfection.

The culmination of this approach is the generation of a heat map, meticulously displaying the UVC light energy distribution across all surfaces traversed by the robot. Consequently, a robust disinfection record is established, facilitating comprehensive tracking and analysis of space disinfection efficacy.

### SUMMARY

This algorithm is specifically tailored for autonomous robots equipped with UVC light emission capabilities. The process is straight forward: upon placing the autonomous mobile robot within a known space, a single start button initiates the algorithm. Utilizing onboard sensors, the robot initially learns a portion of the space to begin movement. Progressing through the environment, the robot continuously maps and expands its coverage to encompass unexplored sections of the space.

While traversing the space, the robot meticulously records the UVC light energy delivered to surfaces in a grid pattern within a user-defined resolution. Movement is directed toward the nearest points within the grid requiring further UVC light exposure. In instances where an area is obstructed, the robot optimally positions itself as close as possible to the obstructed point. Depending on the distance to the obstruction, the robot remains in proximity until the designated light energy level is achieved and extends to adjacent surfaces. This iterative process persists until all spaces are explored, and the entire area has received the configured UVC light energy dosage.

Key distinguishing features of this algorithm encompass:
1. Dynamic Path and Movement Selection: Movement decisions are based on achieving a user-configured UVC light energy threshold on adjacent surfaces.
2. Absence of Pre-existing Maps: No prior spatial map is necessary for effective operation.
3. Space Limitations Considered: In cases of constrained movement, the algorithm calculates the necessary time for emitting the specified UVC light energy to areas within the autonomous mobile robot's line of sight.
4. Automated Heat Map Generation: A comprehensive heat map is automatically generated, visualizing UVC light distribution across all surfaces.

### BRIEF DESCRIPTION OF DRAWINGS

FIG. 1: Sample robot with UVC light that could use the algorithm.

FIG. 2: Workflow diagram to select either a Non-Movement (Stationary) scenario or a Movement scenario.

FIG. 3: Workflow diagram to select goal positions, once the robot has determined it has space to do a movement scenario.

### DETAILED DESCRIPTION

The algorithm was developed to enhance the capabilities of autonomous mobile robots by integrating UVC light emission calculations, to determine the optimal movement to minimize the time required to disinfect a space. The algorithm's primary objective is to address a range of critical challenges inherent to traditional disinfection methods. These challenges encompass human errors associated with conventional disinfection practices, environmental concerns linked to chemical utilization, reduction in the time required to configure intricate mapping procedures for autonomous mobile robots, decreased manual disinfection durations for rooms, streamlined relocation of stationary UVC disinfection devices across multiple positions within a space, and the provision of a comprehensive heat map of the disinfected area for meticulous record-keeping.

This innovation marks a pivotal advancement, as historical disinfection practices have lacked a robust means of systematically tracking and assessing the effectiveness of disinfection processes. The autonomous mobile robot embodies a multifaceted system, replete with an array of sophisticated distance-detecting sensors including two-dimensional lidars, three-dimensional lidars, and sonars. Emitting UVC light with a precisely calibrated irradiance from a predetermined spatial orientation, relative to both the room and the robot, this system can be seamlessly integrated into any given space without the necessity of prior environmental knowledge. The robot rapidly surveys, disinfects, and navigates through the environment, responding dynamically to areas within the room that demand additional UVC light energy for thorough disinfection. Throughout the entirety of these operations, the autonomous mobile robot diligently constructs a comprehensive heat map, recording the distribution of UV light across surfaces throughout the room. This invaluable heat map serves as a tangible metric for evaluating the efficacy of the disinfection process.

To initiate the algorithm, users can effortlessly activate the autonomous mobile robot by means of a single-instance start mechanism. This intuitive initiation can be executed through either a connected application or a physical button. This streamlined commencement process underscores the algorithm's commitment to operational simplicity, facilitating ease of use for operators.

To initiate the algorithm, the autonomous mobile robot activates all distance-based sensors, commencing a comprehensive room scan to detect and identify visible objects within its line of sight. This initial pre-movement scan serves a dual purpose: it not only facilitates spatial awareness but also creates the potential for the robot to optimize its movement path. Following this scan, the robot proceeds with one of two distinct courses of action:

### Obstacle Recognition and Stationary Disinfection:

The algorithm is designed to recognize scenarios in which the available space is insufficient for safe and effective navigation. In such instances, the robot makes a calculated determination to execute UVC light emission from a stationary position. By doing so, the robot ensures comprehensive disinfection in areas it cannot physically access due to limited space.

### Navigational Strategy and Disinfection Cycle:

Upon determining that ample space exists for movement, the robot initiates a recurring cycle of actions. It dynamically evaluates and decides between two strategic pathways:

a. Disinfection-Focused Movement: The robot identifies spaces within the room that necessitate disinfection and strategically maneuvers towards these target areas, ensuring thorough pathogen eradication.

b. Exploration of Uncharted Territories: In parallel with disinfection efforts, the robot ventures towards the frontiers of potential unexplored areas. This dual strategy enables the robot to both optimize disinfection and expand its spatial knowledge.

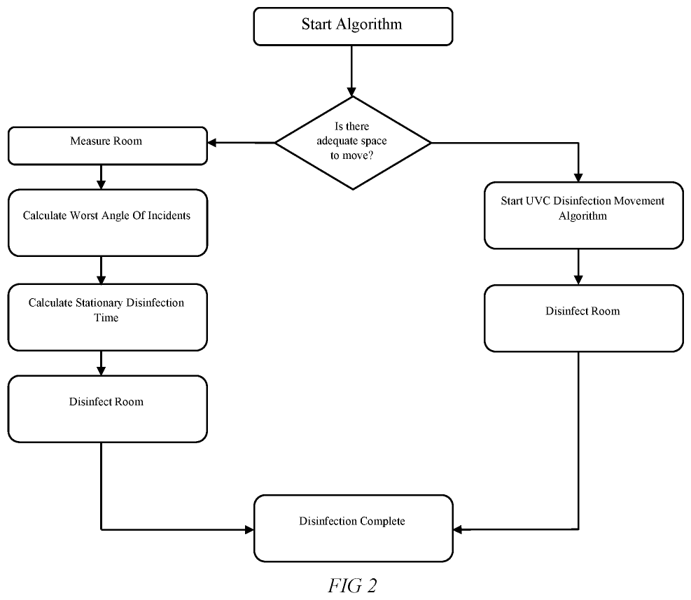

This adaptive decision-making process, shown in FIG. 2, endows the autonomous mobile robot with the ability to intelligently adapt to the unique characteristics of its environment. It seamlessly transitions between stationary disinfection and dynamic movement, resulting in the achievement of optimal and effective disinfection outcomes.

### Stationary Disinfection Scenario:

In the event of the first scenario, the algorithm's complexity is streamlined. The robot commences by measuring the maximum dimensions of the room, enabling the calculation of the requisite UVC light energy needed to effectively reach the furthest distances. This calculation further informs the determination of exposure times necessary for robust disinfection. Throughout this scenario, a dynamic grid-based heat map is continually updated at a predefined frequency. This dynamic update entails the recording of UV light energy levels emitted by the robot, facilitating a comprehensive record of the disinfection process.

### Dynamic Movement and Exploration Scenario:

Conversely, in the second scenario, the algorithm engages in an ongoing process of dynamic movement and exploration. The dynamic grid-based heat map is not only generated but also expands iteratively as the robot navigates through the space. At predetermined intervals, each cell within the grid is updated with the quantified UV light energy that has been successfully delivered to the corresponding section. This iterative process persists until all grid markers attain their configurable desired levels of UV light energy. Subsequently, the robot's progression towards grid markers concludes as all available markers have been addressed.

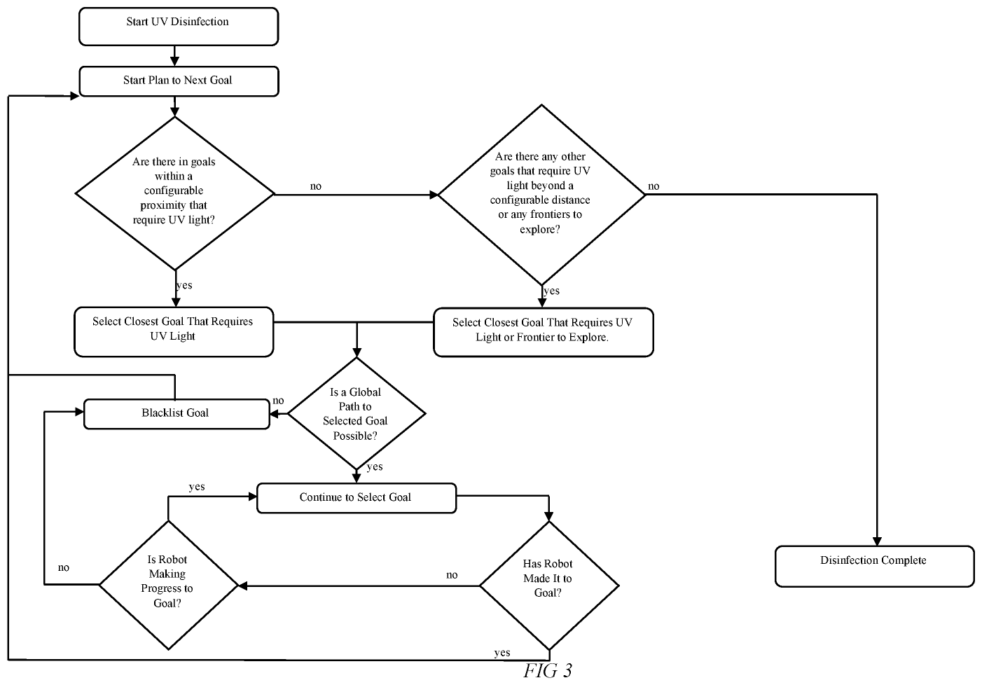

An integral aspect of the algorithm is the harmonization of frontier exploration with UV light energy exploration. The algorithm workflow, shown in FIG. 3 effectively manages the delicate balance between prioritizing movement towards areas necessitating UV light energy and the exploration of uncharted frontiers within the environment. As a general principle, UV light energy grid points, inherently correlated with points on the robot's exploration map, take precedence over frontiers. However, in circumstances where all points within a configurable distance from the robot have reached their desired UV light energy levels, and unexplored frontiers are available, the robot pivots its movement towards these frontiers on the map.

In addition, should there be a grid point, which requires UV energy, and the autonomous mobile robot cannot determine an adequate path to reach the goal, it will pause movement, and allow for a stationary disinfection as close to the unreachable space as it can move.

This strategic interplay between UV light energy optimization and spatial exploration ensures a meticulous and comprehensive disinfection process. The algorithm's innate adaptability enables the autonomous mobile robot to dynamically allocate its efforts towards achieving the most efficient disinfection outcomes, thereby maintaining a harmonious equilibrium between pathogen eradication and spatial expansion.

To quantify the UVC light energy that reaches a surface, it is essential to establish the maximum light energy measured at a distance of one meter from the robot's center. This number becomes a fixed parameter within the algorithm. When surfaces are perfectly perpendicular to the light source, the amount of light reaching these surfaces can be computed using the following equation:

$$Maximum UVC Light Energy at Surface = \frac{Maximum UVC Light Energy Measured at 1 m}{{Distance}^{2}}$$

Here, UVC Light Energy is quantified in millijoules per square centimeter (mJ/cm²), and Distance is measured in meters. Acknowledging that surfaces may not always be perfectly perpendicular, the angle formed between the light source and the surfaces must be considered. At a 90-degree angle to the surface, maximum light energy is achieved, while a 0-degree angle corresponds to minimum light energy.

The UVC light energy that successfully reaches the surface is calculated using the formula:

$$Final UVC Light Energy = Cosine ( ∅ ) \times Maximum UVC Light Energy at Surface$$

In this equation, Ø represents the angle between the source of the maximum UVC light energy and the surface.

It is crucial to note that UVC light necessitates a clear line of sight to access a surface. Consequently, if an object obstructs the view towards a surface and the robot presumes that space exists behind the object, the algorithm refrains from adding the corresponding light energy to the grid map over time.

This quantification of UVC light energy, factoring in distance and angle considerations, ensures a precise determination of the energy delivered to each surface, while also accounting for potential obstructions that might hinder effective disinfection. The algorithm's intricate calculations provide a comprehensive understanding of the disinfection process's spatial dynamics and the UVC light's interaction with surfaces.

Leveraging the intricate 3D mapping accomplished by the robot, an additional dimension is introduced to the surfaces, facilitating meticulous monitoring of the UVC light energy penetration onto each surface. This comprehensive approach affords a multi-faceted understanding of the disinfection process's interaction with the environment.

To streamline movement coordination, the algorithm projects the lowest recorded UVC light energy level onto a 2-dimensional plane that mirrors the robot's designated mapping plane used for spatial navigation. This projection occurs at intervals that can be tailored (typically once per second), ensuring a coherent record of UVC light energy distribution across a two dimensional grid. In this process, each individual grid point is attributed a value expressed in millijoules per square centimeter (UVC Light Energy measurement). The robot maintains vigilant oversight of this grid, persistently progressing towards grid points requiring additional disinfection until the entirety of the space attains the desired disinfection threshold.

The adaptability of the algorithm accommodates variations in the processing power of the autonomous mobile robot. The grid pattern resolution can be adjusted to suit the robot's computational capabilities and the specific disinfection requirements. Ordinarily, a 1 cm grid pattern offers an optimal balance of accuracy and operational efficiency, serving as an effective reference for UVC light energy distribution throughout the space.

This dynamic integration of 3D mapping, 2D projection, and grid-based monitoring ensures a comprehensive and systematic approach to disinfection. By aggregating and analyzing data in multiple dimensions, the algorithm enhances the robot's ability to perform accurate and efficient disinfection operations, thereby contributing to the algorithm's overall effectiveness in diverse environments.

At a user-configurable interval, typically ranging from three to ten seconds, the robot undertakes a meticulous evaluation to ensure precise positioning for achieving the desired UVC disinfection levels. The pattern for selection adheres to a straightforward protocol:

### Grid Point Assessment and Movement Decision:

1) The algorithm initiates by assessing whether any grid points within a user-defined distance (typically one meter) necessitate disinfection.
   a. If affirmative, the robot charts a course towards these identified points requiring disinfection.
   b. If no such points are identified, the algorithm progresses to the next stage.

### Frontier Exploration and Optimal Point Selection:

1) In the absence of immediate disinfection targets, the algorithm investigates unexplored frontiers within the environment.
   a. If uncharted frontiers exist, the robot selects the closest point, whether it is a frontier point or a disinfection point.
   b. In cases where the proximity to both frontier and disinfection points is equivalent, priority is accorded to disinfection points.
   c. If no viable frontiers or disinfection points are present, the algorithm concludes that the disinfection process is complete.

When faced with a situation wherein a grid point lies beyond the robot's immediate reach, the autonomous mobile robot undertakes a proactive approach to get as close as feasible, adhering to a user-defined distance, which is typically set at two meters by default. The robot then maintains a stationary position, abstaining from movement, until the desired UVC light levels effectively reach the surfaces within the designated distance. Should circumstances arise where the robot cannot attain the prescribed proximity or establish a direct line of sight to the target surface, the resulting disinfection heat map will indicate an untreated area, underscoring the algorithm's meticulous record-keeping of the disinfection process.

This iterative process of identifying disinfection points and exploring frontiers (see FIG. 3) persists until the algorithm determines that no remaining grid points necessitate disinfection. This iterative approach ensures that every relevant section within the space undergoes comprehensive disinfection, while simultaneously fostering exploration in uncharted territories.

This innovation represents a paradigm shift within the realm of autonomous mobile robots, transcending traditional exploration algorithms. The distinctive feature of this invention lies in its utilization of intricate equations governing light energy absorption on surfaces, thereby enabling the algorithm to make calculated decisions regarding movement, pauses, and the achievement of exploration objectives. Furthermore, a pivotal outcome of this algorithmic process is the generation of comprehensive heat maps that elucidate the distribution of UV Light Energy throughout the designated space.

The significance of this advancement resonates profoundly across industries that place a premium on rigorous disinfection standards. Conventional indicators of cleanliness, such as sensory cues or visual assessments, often fall short of providing a comprehensive validation of disinfection efficacy. However, the integration of intricate heat maps into the algorithm's output ushers in an era of empirical validation. These heat maps establish a quantifiable link between the eradication of pathogens on surfaces and the precise application of UV Light dosage.

The efficacy of the algorithm hinges on the capabilities of the autonomous mobile robot, necessitating traits of agility and nimbleness. These attributes enable the robot to traverse complex spaces with precision. The UV light source, strategically aligned with practical considerations, maintains a stature harmonious with typical door heights, ensuring thorough and efficient coverage. Moreover, the demand for sustained operation necessitates robust power reserves, typically exceeding 1000 Watt Hours, to facilitate extended disinfection processes. The autonomous mobile robot is further equipped with distance-reading sensors, such as lidars or sonars, which contribute to the creation of accurate environmental representations in the form of point clouds or lidar scans. These sensor-driven insights enable the robot to navigate and execute its tasks with unwavering accuracy.

A practical illustration of the algorithm's efficacy lies in its application within hospital rooms. In a domain where manual disinfection practices are time-consuming and prone to human error, the collaboration between the algorithm and the UV Light Energy Emitting-capable autonomous mobile robot streamlines the process. Disinfection durations that once spanned from 20 minutes to 2 hours can now be significantly reduced by up to twenty times quicker, all without human intervention. Furthermore, the algorithm's ability to generate heat maps following each disinfection cycle offers tangible documentation of the disinfection outcome. This empirical evidence elevates record-keeping from a mere formality to a scientific demonstration of efficacy, offering heightened assurance to healthcare facilities and other sensitive spaces.

This innovation fuses technological ingenuity with practical application, recalibrating established disinfection paradigms. Through the symbiotic interplay of technology and method, a transformational narrative unfolds-one characterized by precision, validation, and a resolute commitment to a future free from pathogens.

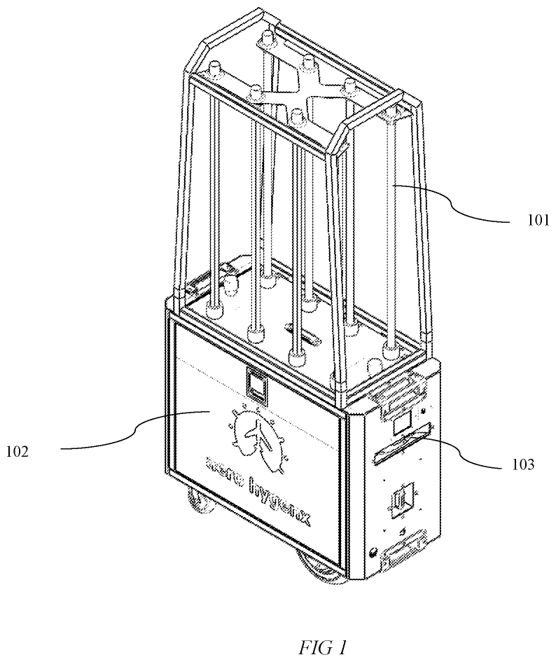

With reference to the figures, the following table describes the elements within:

| | Name | Description |
|---|---|---|
| FIG. 1 | Sample | A sample autonomous mobile robot, that can emit |
| | Robot | UVC light energy. |
| 101 | UVC | The lamps on board the autonomous mobile robot |
| | Lamps | that emits the UVC light. |
| 102 | Robot | The portion of the autonomous mobile robot, that |
| | Base | houses all of the electronics, batteries and robotic |
| | | sensors. |
| 103 | Sample | A sample distance measuring sensor that can be |
| | Sensor | used with the algorithm. |
| FIG. 2 | Stationary | This workflow, shows the decision making |
| | or Motion | workflow between the autonomous mobile robot |
| | Decision | being put into motion and maintaining a stationary |
| | Making | position for UV light emission. |
| | Workflow | |
| FIG. 3 | Grid Point | This workflow, shows the decisions made by the |
| | Selection | algorithm to select goal points for the autonomous |
| | workflow | mobile robot to move towards. |

The invention claimed is:

1. An adaptive decision-making module that evaluates available space, determining the most suitable mode of operation between stationary UVC light emission and dynamic light emission through movement.

2. A movement control algorithm that guides the robot towards spaces requiring disinfection and uncharted territories, orchestrating efficient and comprehensive coverage.

3. A real-time heat map generation component that constructs and updates a dynamic grid-based heat map, illustrating the spatial distribution of UV light energy on surfaces.
